(19) United States
(12) Patent Application Publication
(10) Pub. No.: US 2023/0270643 A1
(43) Pub. Date: Aug. 31, 2023

(54) DYEING AND/OR LIGHTENING COMPOSITION COMPRISING AN ANTHRAQUINONE CATIONIC DIRECT DYE, A FATTY SUBSTANCE, A CHEMICAL OXIDIZING AGENT AND A BASIFYING AGENT AND/OR AN OXIDATION DYE PRECURSOR

(71) Applicant: L'Oreal, Paris (FR)

(72) Inventors: Valérie NICOU, SAINT-OUEN (FR); Samira RHARBI, SAINT-OUEN (FR); Juilie BLANC, SAINT-OUEN (FR)

(21) Appl. No.: 18/008,250

(22) PCT Filed: Jun. 29, 2021
(86) PCT No.: PCT/EP2021/067823

§ 371 Date: Dec. 5, 2022

(30) Foreign Application Priority Data
Jun. 30, 2020 (FR) FR2006859

(51) Int. Cl. A61K 8/41; A61K 8/92; A61K 8/22; A61Q 5/10; A61Q 5/08

(52) U.S. Cl. CPC A61K 8/41; A61K 8/22; A61K 8/92; A61Q 5/08; A61Q 5/10; A61K 2800/88; A61K 2800/4324

(57) ABSTRACT

The invention relates to a composition for dyeing and/or lightening human keratin fibers, preferably the hair, comprising:
one or more anthraquinone cationic direct dyes,
at least 25% of one or more fatty substances,
one or more chemical oxidizing agents,
one or more basifying agents and/or one or more oxidation dye precursors, preferably one or more oxidation dye precursors.

One subject of the present invention is a composition for dyeing and/or lightening human keratin fibers, notably the hair, comprising an anthraquinone cationic direct dye, a fatty substance in a particular content, a chemical oxidizing agent and a basifying agent and/or an oxidation dye precursor. The invention also relates to a dyeing process using this composition.

Many people have sought for a long time to modify the color of their hair.

A first dyeing method, commonly referred to as semi-permanent dyeing or direct dyeing, consists in applying, to the keratin fibers, direct dyes, which are colored and coloring molecules that have an affinity for said fibers.

These dyes are usually applied to the fibers, optionally in the presence of a chemical oxidizing agent if it is desired to obtain simultaneous lightening of the fibers. Once the leave-on time has elapsed, the fibers are rinsed, optionally washed and dried.

The colorings that result therefrom are generally chromatic colorings that are, however, temporary or semi-permanent since the nature of the interactions that bind the direct dyes to the keratin fiber and their desorption from the surface and/or the core of the fiber are responsible for their weaker dyeing power and their poorer persistence with respect to washing or perspiration.

A second dyeing method has therefore been developed thus enabling a long-lasting coloring of the keratin fibers. This second method, also referred to as permanent dyeing or oxidation dyeing, uses dye compositions containing oxidation bases. These oxidation bases are colorless or weakly colored compounds which, when combined with oxidizing products, may give rise to colored compounds via a process of oxidative condensation.

It is also possible to vary the shades obtained with these oxidation bases by combining them with couplers or color modifiers. The variety of molecules used as oxidation bases and couplers allows a wide range of colors to be obtained.

To further vary the shades obtained and give them tints, it is known to use, in combination with the oxidation bases and the couplers, direct dyes.

Furthermore, it is known to lighten the hair in the presence of a persalt (also known as a peroxygenated salt), in particular persulfate, an alkaline agent and hydrogen peroxide when it is desired to achieve higher levels of lightening. However, these bleaching treatments are generally accompanied by the appearance of unattractive orange-yellow tints. Direct dyes may then be introduced into the bleaching compositions to obtain a more attractive color rendition.

However, direct dyes show little or no resistance to chemical oxidizing agents. This poor stability of direct dyes with respect to oxidation especially has the consequence of producing hair colorings that have poor color build-up and a low level of lightening of the keratin fibers.

It is especially difficult to obtain powerful and/or persistent colorings for certain shades, especially blue shades, in particular at alkaline pH values.

Furthermore, consumers are still in search of dye compositions that exhibit optimum usage qualities.

The objective of the present invention is to provide a dyeing and/or lightening composition exhibiting improved usage qualities and leading to good dyeing properties.

In particular, one of the objectives of the present invention is to provide compositions that exhibit good lightening performance and/or that make it possible to obtain a coloring with varied shades, especially shades that are natural, attractive, powerful, sparingly selective and/or that show good resistance to the various attacks to which the hair may be subjected, in particular shampoo washes.

This objective is achieved by the present invention, one subject of which is especially a composition for dyeing and/or lightening keratin fibers such as the hair, comprising:
one or more anthraquinone cationic direct dyes,
at least 25% by weight of one or more fatty substances relative to the total weight of the composition,
one or more chemical oxidizing agents, and
one or more alkaline agents and/or one or more oxidation dye precursors, preferably one or more oxidation dye precursors.

By formulating these direct dyes of anthraquinone structure in this specific support, it is possible to obtain a composition that is easy to apply to the hair, without running, while having good dyeing properties. The composition obtained also makes it possible to obtain natural shades and a coloring that bleeds little, in particular after the final shampoo wash. They may therefore be rinsed rapidly, without using a large amount of water.

Another subject of the invention is a process for dyeing and/or lightening human keratin fibers, preferably the hair, wherein the composition as defined above is applied to the keratin fibers.

Another subject of the invention is a multi-compartment device, each compartment comprising a composition, the compositions being mixed at the moment of use in order to obtain the composition according to the invention.

Other subjects, features, aspects and advantages of the invention will emerge even more clearly on reading the description and the examples that follow.

In the text hereinbelow, and unless otherwise indicated, the limits of a range of values are included in that range, notably in the expressions “between” and “ranging from ... to ...”.

Moreover, the expression “at least one” used in the present description is equivalent to the expression “one or more”.

### ANTHRAQUINONE CATIONIC DIRECT DYE(S)

The composition according to the invention comprises at least one anthraquinone cationic direct dye.

A “cationic direct dye” is understood to mean any dye other than oxidation dyes, commonly referred to as “basic” direct dyes or “basic dyes”. They are referred to as “basic” by virtue of their affinity for acidic substances, notably including at least one cationic or cationizable endocyclic or exocyclic group within their structure.

Preferably, the cationic direct dye(s) is (are) synthetic, namely obtained solely by chemical synthesis and is (are) not naturally occurring. In particular, it is a dye which is not obtained from a plant matrix or by fermentation and which diffuses superficially on the fiber.

The cationic direct dye(s) that may be used in the composition according to the invention comprise a quaternary ammonium group.

These cationic radicals are for example a cationic radical:
bearing a (C₁-C₈)alkylammonium exocyclic charge, or
bearing an endocyclic charge, such as comprising a cationic heteroaryl group chosen from: acridinium, benzimidazolium, benzobistriazolium, benzopyrazolium, benzopyridazinium, benzoquinolium, benzothiazolium, benzotriazolium, benzoxazolium, bipyridinium, bistetrazolium, dihydrothiazolium, imidazopyridinium, imidazolium, indolium, isoquinolium, naphthoimidazolium, naphthoxazolium, naphthopyrazolium, oxadiazolium, oxazolium, oxazolopyridinium, oxonium, phenazinium, phenooxazolium, pyrazinium, pyrazolium, pyrazoyltriazolium, pyridinium, pyridinoimidazolium, pyrrolium, pyrylium, quinolium, tetrazolium, thiadiazolium, thiazolium, thiazolopyridinium, thiazoylimidazolium, thiopyrylium, triazolium or xanthylium.

Preferably, the cationic charge is exocyclic.

Among the anthraquinone cationic direct dyes, those of formula (I) having an exocyclic cationic charge are preferred:

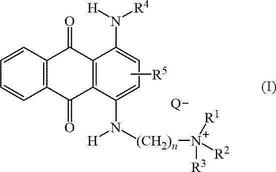

wherein:
R¹, R² and R³, which may be identical or different, representing a hydrogen atom or an optionally substituted (C₁-C₈)alkyl group;
R⁴ representing a hydrogen atom or an optionally substituted (C₁-C₈)alkyl group;
R⁵ representing a hydrogen atom, an optionally substituted (C₁-C₈)alkyl group, an optionally substituted (C₁-C₈)alkylene group, a halogen, a hydroxyl group or a (C₁-C₈)alkoxy group;
n representing a number between 1 and 8;
Q⁻ represents an organic or mineral anionic counterion, such as a halide or an alkyl sulfate.

Preferably, in formula (I):
R¹, R² and R³, which may be identical or different, representing an optionally substituted (C₁-C₆)alkyl group;
R⁴ representing a hydrogen atom or optionally substituted (C₁-C₆)alkyl group;
R⁵ representing a hydrogen atom or an optionally substituted (C₁-C₈)alkyl group;
n representing a number between 1 and 6;
Q⁻ representing a halide or an alkyl sulfate.

More preferentially, in formula (I):
R¹, R² and R³, which may be identical or different, representing an optionally substituted (C₁-C₃)alkyl group;
R⁴ representing a hydrogen atom or a methyl, preferably a methyl;
R⁵ representing a hydrogen atom or a methyl, preferably a hydrogen atom;
n representing a number between 2 and 4;
Q⁻ representing a halide or an alkyl sulfate, preferably a halide.

Among the dyes of formula (I), use may in particular be made of the dyes of formula (I′) or of formula (I″) below:

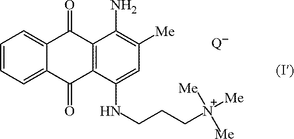

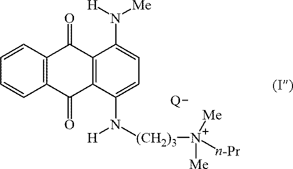

with Q⁻ an anionic counterion, particularly a halide such as bromide or chloride, or an alkyl sulfate, such as methyl sulfate or mesityl. Preferably, Q⁻ is a halide, better still a bromide.

The term “anionic counterion” is intended to mean an anion or an anionic group derived from an organic or mineral acid salt which counterbalances the cationic charge of the dye; more particularly, the anionic counterion is chosen from i) halides such as chloride or bromide; ii) nitrates; iii) sulfonates, including C₁-C₆ alkylsulfonates: Alk-S(O)₂O⁻ such as methanesulfonate or mesylate, and ethanesulfonate; iv) arylsulfonates: Ar-S(O)₂O⁻ such as benzenesulfonate and toluenesulfonate or tosylate; v) citrate; vi) succinate; vii) tartrate; viii) lactate; ix) alkyl sulfates: Alk-O-S(O)O⁻ such as methyl sulfate and ethyl sulfate; x) aryl sulfates: Ar-O-S(O)O⁻ such as benzene sulfate and toluene sulfate; xi) alkoxy sulfates: Alk-O-S(O)₂O⁻ such as methoxy sulfate and ethoxy sulfate; xii) aryloxy sulfates: Ar-O-S(O)₂O⁻; xiii) phosphates O=P(OH)₂-O⁻, O=P(O⁻)₂-OH, O=P(O⁻)₃, HO-[P(O)(O⁻)]_{w}-P(O)(O⁻)₂ with w being an integer; xiv) acetate; xv) triflate; and xvi) borates such as tetrafluoroborate; xvii) disulfate (O=)₂S(O⁻)₂ or SO₄²⁻ and monosulfate HSO₄⁻.

One particularly preferred dye of formula (I′) is HC Blue 17.

One particularly preferred dye of formula (I″) is HC Blue 16 (1-methylamino-4-(3′-dimethylpropylammoniumpropylamino)anthraquinone bromide).

Preferably, the anthraquinone cationic direct dye(s) of formula (I) is (are) chosen from the dyes of formula (I′) and (I″) and mixtures thereof, and preferentially from the dyes of formula (I″).

Preferably, the anthraquinone cationic direct dye(s) of formula (I) is (are) chosen from HC Blue 16, HC Blue 17, or mixtures thereof, more preferentially HC Blue 16.

According to another embodiment, the anthraquinone cationic direct dyes are chosen from the dyes of formula (II):

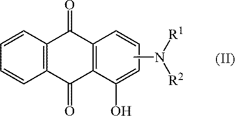

wherein:
R¹ and R², which may be identical or different, representing a hydrogen atom, an optionally substituted (C₁-C₈)alkyl group, an optionally substituted (C₁-C₈)alkylene group, a halogen, a hydroxyl group or a (C₁-C₈)alkoxy group.

Preferably, in formula (II):
R¹ representing a hydrogen atom;
R² representing a (C₁-C₆)alkyl group substituted with a 5-, 6- or 7-membered heterocycle comprising at least one nitrogen atom, one oxygen atom or one sulfur atom.

More preferentially, in formula (II):
R¹ representing a hydrogen atom;
R² representing a (C₁-C₆)alkyl group substituted with a morpholinyl or alkylmorpholinyl group.

More preferentially, use will be made of the dyes of formula (II′) below:

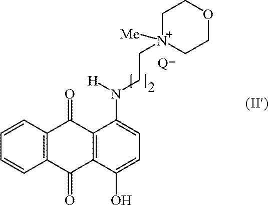

with Q⁻ an anionic counterion as defined previously, particularly a halide, such as bromide or chloride, or an alkyl sulfate, such as methyl sulfate or mesityl. Preferably, Q⁻ is an alkyl sulfate, better still a methyl sulfate.

The dye corresponding to formula (II′) is preferably 1-(N-methylmorpholiniumpropylamino)-4-hydroxyanthraquinone methyl sulfate.

Preferentially, the anthraquinone cationic direct dye(s) is (are) chosen from the dyes of formula (I′) and (I″), and more preferentially from the dyes of formula (I″), better still from HC Blue 16.

Advantageously, the total amount of anthraquinone cationic direct dye(s) varies from 0.01% to 15% by weight, preferably from 0.05% to 10% by weight, more preferentially from 0.1% to 5% by weight, better still from 0.3% to 3% by weight relative to the total weight of the composition.

Advantageously, the total amount of anthraquinone cationic direct dye(s) of formula (I) varies from 0.01% to 15% by weight, preferably from 0.05% to 10% by weight, more preferentially from 0.1% to 5% by weight, better still from 0.3% to 3% by weight relative to the total weight of the composition.

Advantageously, the total amount of anthraquinone cationic direct dye(s) of formula (I″) varies from 0.01% to 15% by weight, preferably from 0.05% to 10% by weight, more preferentially from 0.1% to 5% by weight, better still from 0.3% to 3% by weight relative to the total weight of the composition.

### FATTY SUBSTANCE

The composition according to the invention comprises at least 25% of one or more fatty substances.

The term “fatty substance” is intended to mean an organic compound that is insoluble in water at ordinary room temperature (25° C.) and at atmospheric pressure (760 mmHg) (solubility of less than 5%, preferably less than 1% and even more preferentially less than 0.1%). They bear in their structure at least one hydrocarbon-based chain comprising at least 6 carbon atoms or a sequence of at least two siloxane groups. In addition, the fatty substances are generally soluble in organic solvents under the same temperature and pressure conditions, for instance chloroform, ethanol, benzene, liquid petroleum jelly or decamethylcyclopentasiloxane.

These fatty substances are neither polyoxyethylenated nor polyglycerolated. They are different from fatty acids since salified fatty acids constitute soaps which are generally soluble in aqueous media.

The fatty substances are in particular chosen from C₆-C₁₆ hydrocarbons or hydrocarbons comprising more than 16 carbon atoms and in particular alkanes, oils of animal origin, oils of plant origin, glycerides or fluoro oils of synthetic origin, fatty alcohols, fatty acid and/or fatty alcohol esters, non-silicone waxes, and silicones.

It is recalled that, for the purposes of the invention, the fatty alcohols, fatty esters and fatty acids more particularly contain one or more linear or branched, saturated or unsaturated hydrocarbon-based groups comprising 6 to 30 carbon atoms, which are optionally substituted, in particular with one or more (in particular 1 to 4) hydroxyl groups. If they are unsaturated, these compounds may comprise one to three conjugated or unconjugated carbon-carbon double bonds.

As regards the C₆-C₁₆ alkanes, they are linear or branched, and possibly cyclic. Examples that may be mentioned include hexane, dodecane and isoparaffins such as isohexadecane and isodecane. The linear or branched hydrocarbons containing more than 16 carbon atoms may be chosen from liquid paraffins, petroleum jelly, liquid petroleum jelly, polydecenes, and hydrogenated polyisobutene such as Parleam®.

The fatty substance(s) used in the composition of the invention may be chosen from volatile linear alkanes.

The term “volatile linear alkane” is intended to mean, without distinction, “a volatile linear alkane oil”.

A volatile linear alkane that is suitable for the invention is liquid at room temperature (about 25° C.) and under atmospheric pressure (101 325 Pa or 760 mmHg).

The term “volatile linear alkane” that is suitable for the invention is intended to mean a linear alkane that can evaporate on contact with the skin in less than one hour, at room temperature (25° C.) and under atmospheric pressure (101 325 Pa), which is liquid at room temperature, in particular having an evaporation rate ranging from 0.01 to 15 mg/cm²/minute, at room temperature (25° C.) and under atmospheric pressure (101 325 Pa).

Preferably, the volatile linear alkanes that are suitable for the invention have an evaporation rate ranging from 0.01 to 3.5 mg/cm²/minute, better still from 0.01 to 1.5 mg/cm²/minute, at room temperature (25° C.) and under atmospheric pressure (101 325 Pa).

More preferably, the volatile linear alkanes that are suitable for the invention have an evaporation rate ranging from 0.01 to 0.8 mg/cm²/minute, preferentially from 0.01 to 0.3 mg/cm²/minute, and even more preferentially from 0.01 to 0.12 mg/cm²/minute, at room temperature (25° C.) and under atmospheric pressure (101 325 Pa).

The evaporation rate of a volatile alkane in accordance with the invention (and more generally of a volatile solvent) may in particular be evaluated by means of the protocol described in WO 06/013413, and more particularly by means of the protocol described below.

15 g of volatile hydrocarbon-based solvent are introduced into a crystallizing dish (diameter: 7 cm) placed on a balance that is in a chamber of about 0.3 m³ which is temperature-regulated (25° C.) and hygrometry-regulated (50% relative humidity).

The volatile hydrocarbon-based solvent is allowed to evaporate freely, without stirring it, while providing ventilation by means of a fan (Papst-Motoren, reference 8550 N, rotating at 2700 rpm) placed in a vertical position above the crystallizing dish containing the volatile hydrocarbon-based solvent, the blades being directed toward the crystallizing dish, 20 cm away from the bottom of the crystallizing dish.

The mass of volatile hydrocarbon-based solvent remaining in the crystallizing dish is measured at regular time intervals.

The evaporation profile of the solvent is then obtained by plotting the curve of the amount of product evaporated (in mg/cm²) as a function of the time (in minutes).

The evaporation rate is then calculated, which corresponds to the tangent to the origin of the curve obtained. The evaporation rates are expressed in mg of volatile solvent evaporated per unit area (cm²) and per unit time (minutes).

Preferably, the volatile linear alkanes that are suitable for the invention have a non-zero vapor pressure (also known as the saturation vapor pressure), at room temperature, in particular a vapor pressure ranging from 0.3 Pa to 6000 Pa.

Preferably, the volatile linear alkanes that are suitable for the invention have a vapor pressure ranging from 0.3 to 2000 Pa, better still from 0.3 to 1000 Pa, at room temperature (25° C.).

More preferably, the volatile linear alkanes that are suitable for the invention have a vapor pressure ranging from 0.4 to 600 Pa, preferentially from 1 to 200 Pa, and more preferentially still from 3 to 60 Pa, at room temperature (25° C.).

A volatile linear alkane that is suitable for the invention may have a flash point that is within the range from 30° C. to 120° C., and more particularly from 40° C. to 100° C. The flash point is in particular measured according to standard ISO 3679.

According to one embodiment, the volatile linear alkanes that are suitable in the invention may be linear alkanes comprising from 7 to 15 carbon atoms, preferably from 8 to 14 carbon atoms and better still from 9 to 14 carbon atoms.

More preferably, the volatile linear alkanes that are suitable in the invention may be linear alkanes comprising from 10 to 14 carbon atoms, and more preferentially still from 11 to 14 carbon atoms.

A volatile linear alkane that is suitable for the invention may advantageously be of plant origin.

Preferably, the volatile linear alkane or the mixture of volatile linear alkanes present in the composition according to the invention comprises at least one ¹⁴C (carbon-14) carbon isotope. In particular, the ¹⁴C isotope may be present in a ¹⁴C/¹²C isotope ratio by number (or ¹⁴C/¹²C ratio) of greater than or equal to 1×10⁻¹⁶, preferably greater than or equal to 1×10⁻¹⁵, more preferably greater than or equal to 7.5×10⁻¹⁴, and better still greater than or equal to 1.5×10⁻¹³. Preferably, the ¹⁴C/¹²C ratio ranges from 6×10⁻¹³ to 1.2×10⁻¹².

The amount of ¹⁴C isotopes in the volatile linear alkane or the mixture of volatile linear alkanes may be determined via methods known to those skilled in the art such as the Libby counting method, liquid scintillation spectrometry or else accelerator mass spectrometry.

Such an alkane may be obtained, directly or in several steps, from a plant raw material, such as an oil, a butter, a wax, etc.

As examples of alkanes that are suitable for the invention, mention may be made of the alkanes described in patent applications WO 2007/068371 and WO 2008/155059. These alkanes are obtained from fatty alcohols, which are themselves obtained from coconut kernel oil or palm oil.

As examples of linear alkanes that are suitable for the invention, mention may be made of n-heptane (C7), n-octane (C8), n-nonane (C9), n-decane (C10), n-undecane (C11), n-dodecane (C12), n-tridecane (C13), n-tetradecane (C14), and n-pentadecane (C15), and mixtures thereof. According to a particular embodiment, the volatile linear alkane is chosen from n-nonane, n-undecane, n-dodecane, n-tridecane and n-tetradecane, and mixtures thereof.

According to one preferred embodiment, mention may be made of the mixtures of n-undecane (C11) and of n-tridecane (C13) obtained in Examples 1 and 2 of application WO 2008/155059.

Mention may also be made of n-dodecane (C12) and n-tetradecane (C14) sold, respectively, under the references Parafol 12-97 and Parafol 14-97 by the company Sasol, and also mixtures thereof.

One embodiment consists in using only one volatile linear alkane.

Alternatively, a mixture of at least two different volatile linear alkanes, differing from one another by a carbon number n of at least 1, in particular differing from one another by a carbon number of 1 or 2, may be used.

According to one embodiment, a mixture of at least two different volatile linear alkanes comprising from 10 to 14 carbon atoms and differing from one another by a carbon number of at least 1 is used. Examples that may especially be mentioned include the mixtures of C10/C11, C11/C12 or C12/C13 volatile linear alkanes.

According to another embodiment, a mixture of at least two different volatile linear alkanes comprising from 10 to 14 carbon atoms and differing from one another by a carbon number of at least 2 is used. Examples that may especially be mentioned include the mixtures of C10/C12, or C12/C14, volatile linear alkanes, for an even carbon number n and the C11/C13 mixture for an odd carbon number n.

According to a preferred embodiment, a mixture of at least two different volatile linear alkanes comprising from 10 to 14 carbon atoms and differing from one another by a carbon number of at least 2, and in particular a mixture of C11/C13 volatile linear alkanes or a mixture of C12/C14 volatile linear alkanes, is used.

Other mixtures combining more than two volatile linear alkanes according to the invention, for instance a mixture of at least three different volatile linear alkanes comprising from 7 to 15 carbon atoms and differing from one another by a carbon number of at least 1, may be used in the invention.

In the case of mixtures of two volatile linear alkanes, said two volatile linear alkanes preferably represent more than 95% and better still more than 99% by weight of the mixture.

According to a particular embodiment of the invention, in a mixture of volatile linear alkanes, the volatile linear alkane having the smallest carbon number is predominant in the mixture.

According to another embodiment of the invention, a mixture of volatile linear alkanes in which the volatile linear alkane having the largest carbon number is predominant in the mixture is used.

As examples of mixtures that are suitable for the invention, mention may be made especially of the following mixtures:
from 50% to 90% by weight, preferably from 55% to 80% by weight, more preferentially from 60% to 75% by weight of a Cn volatile linear alkane with n ranging from 7 to 15,
from 10% to 50% by weight, preferably from 20% to 45% by weight, preferably from 24% to 40% by weight of a Cn+x volatile linear alkane with x greater than or equal to 1, preferably x = 1 or x = 2, with n+x between 8 and 14, relative to the total weight of the alkanes in said mixture.

In particular, said mixture of volatile linear alkanes may also contain:
less than 2% by weight and preferably less than 1% by weight of branched hydrocarbons,
and/or less than 2% by weight and preferably less than 1% by weight of aromatic hydrocarbons,
and/or less than 2% by weight, preferably less than 1% by weight and preferentially less than 0.1% by weight of unsaturated hydrocarbons, said percentages being expressed relative to the total weight of the mixture.

More particularly, the volatile linear alkanes that are suitable in the invention may be used in the form of an n-undecane/n-tridecane mixture.

In particular, use will be made of a mixture of volatile linear alkanes comprising:
from 55% to 80% by weight and preferably from 60% to 75% by weight of C11 volatile linear alkane (n-undecane) and
from 20% to 45% by weight and preferably from 24% to 40% by weight of C13 volatile linear alkane (n-tridecane), relative to the total weight of the alkanes in said mixture.

According to one particular embodiment, the mixture of alkanes is an n-undecane/n-tridecane mixture. In particular, such a mixture may be obtained according to Example 1 or Example 2 of application WO 2008/155059.

According to another particular embodiment, the n-dodecane sold under the reference Parafol 12-97 by Sasol is used.

According to another particular embodiment, the n-tetradecane sold under the reference Parafol 14-97 by Sasol is used.

According to yet another embodiment, a mixture of n-dodecane and n-tetradecane is used.

Among the animal oils, mention may be made of perhydrosqualene.

Among the triglycerides of plant or synthetic origin, mention may be made of liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, sunflower oil, castor oil, avocado oil, jojoba oil, shea butter oil, and caprylic/capric acid triglycerides, for instance those sold by the company Stearinerie Dubois or those sold under the names Miglyol® 810, 812 and 818 by the company Dynamit Nobel.

Fluoro oils that may be mentioned include perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or alternatively bromoperfluorooctyl sold under the name Foralkyl® by the company Atochem; nonafluoromethoxybutane and nonafluoroethoxyisobutane; perfluoromorpholine derivatives such as 4-trifluoromethyl perfluoromorpholine sold under the name PF 5052® by the company 3M.

The fatty alcohols that may be used in the compositions of the invention are saturated or unsaturated, linear or branched, and comprise from 6 to 30 carbon atoms and more particularly from 8 to 30 carbon atoms. Examples that may be mentioned include cetyl alcohol, stearyl alcohol and the mixture thereof (cetylstearyl alcohol), octyldodecanol, 2-butyloctanol, 2-hexyldecanol, 2-undecylpentadecanol, oleyl alcohol and linoleyl alcohol.

The wax(es) that may be used in the compositions of the invention are chosen especially from carnauba wax, candelilla wax, esparto grass wax, paraffin wax, ozokerite, plant waxes, for instance olive tree wax, rice wax, hydrogenated jojoba wax or the absolute waxes of flowers such as the essential wax of blackcurrant blossom sold by the company Bertin (France), animal waxes, for instance beeswaxes, or modified beeswaxes (cerabellina); other waxes or waxy starting materials that may be used according to the invention are especially marine waxes such as the product sold by the company Sophim under the reference M82, and polyethylene waxes or polyolefin waxes in general.

As regards the esters of fatty acids and/or of fatty alcohols, advantageously different from the triglycerides mentioned above, mention may be made especially of esters of saturated or unsaturated, linear or branched C1-C26 aliphatic mono- or polyacids and of saturated or unsaturated, linear or branched C1-C26 aliphatic mono-or polyalcohols, the total carbon number of the esters more particularly being greater than or equal to 10.

Among the monoesters, mention may be made of dihydroabietyl behenate; octyldodecyl behenate; isocetyl behenate; cetyl lactate; C12-C15 alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, esters of C4-C22 dicarboxylic or tricarboxylic acids and of C1-C22 alcohols and esters of monocarboxylic, dicarboxylic or tricarboxylic acids and of C2-C26 dihydroxy, trihydroxy, tetrahydroxy or pentahydroxy alcohols may also be used.

Mention may notably be made of: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, it is preferred to use ethyl, isopropyl, myristyl, cetyl or stearyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate or cetyl octanoate.

The composition may also comprise, as fatty ester, sugar esters and diesters of C6-C30 and preferably C12-C22 fatty acids. It is recalled that the term “sugar” refers to oxygen-bearing hydrocarbon-based compounds bearing several alcohol functions, with or without aldehyde or ketone functions, and which include at least 4 carbon atoms. These sugars may be monosaccharides, oligosaccharides or polysaccharides.

Examples of suitable sugars that may be mentioned include sucrose, glucose, galactose, ribose, fucose, maltose, fructose, mannose, arabinose, xylose and lactose, and derivatives thereof, notably alkyl derivatives, such as methyl derivatives, for instance methylglucose.

The sugar esters of fatty acids may be notably chosen from the group comprising the esters or mixtures of esters of sugars described previously and of linear or branched, saturated or unsaturated C6-C30 and preferably C12-C22 fatty acids. If they are unsaturated, these compounds may comprise one to three conjugated or unconjugated carbon-carbon double bonds.

The esters according to this variant may also be chosen from mono-, di-, tri-and tetraesters, polyesters, and mixtures thereof.

These esters may be, for example, oleates, laurates, palmitates, myristates, behenates, cocoates, stearates, linoleates, linolenates, caprates and arachidonates, or mixtures thereof such as, notably, oleopalmitate, oleostearate and palmitostearate mixed esters.

More particularly, use is made of monoesters and diesters and notably sucrose, glucose or methylglucose monooleate or dioleate, stearate, behenate, oleopalmitate, linoleate, linolenate or oleostearate.

An example that may be mentioned is the product sold under the name Glucate® DO by the company Amerchol, which is a methylglucose dioleate.

Examples of esters or mixtures of esters of sugar and of fatty acid that may also be mentioned include:
the products sold under the names F160, F140, F110, F90, F70 and SL40 by the company Crodesta, respectively denoting sucrose palmitostearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;
the products sold under the name Ryoto Sugar Esters, for example referenced B370 and corresponding to sucrose behenate formed from 20% monoester and 80% diester-triester-polyester;
the sucrose monopalinitostearate-dipalmitostearate sold by the company Goldschmidt under the name Tegosoft® PSE.

The silicones that may be used in the composition of the present invention are volatile or non-volatile, cyclic, linear or branched silicones, which are unmodified or modified with organic groups, having a viscosity from 5×10⁻⁶ to 2.5 m²/s at 25° C., and preferably 1×10⁻⁵ to 1 m²/s.

The silicones that may be used in accordance with the invention may be in the form of oils, waxes, resins or gums, preferably silicone oils.

Preferably, the silicone is chosen from polydialkylsiloxanes, in particular polydimethylsiloxanes (PDMSs), and organomodified polysiloxanes comprising at least one functional group chosen from poly(oxyalkylene) groups, amino groups and alkoxy groups.

Organopolysiloxanes are defined in greater detail in WalterNoll’s “Chemistry and Technology of Silicones” (1968), Academic Press. They may be volatile or nonvolatile.

When they are volatile, the silicones are more particularly chosen from those with a boiling point of between 60° C. and 260° C., and even more particularly from:

cyclic polydialkylsiloxanes comprising from 3 to 7 and preferably from 4 to 5 silicon atoms. These are, for example, octamethylcyclotetrasiloxane notably sold under the name Volatile Silicone® 7207 by Union Carbide or Silbione® 70045 V2 by Rhodia, decamethylcyclopentasiloxane sold under the name Volatile Silicone® 7158 by Union Carbide, and Silbione® 70045 V5 by Rhodia, and mixtures thereof.

Mention may also be made of cyclocopolymers of the dimethylsiloxane/methylalkylsiloxane type, such as Silicone Volatile® FZ 3109 sold by the company Union Carbide, of formula:

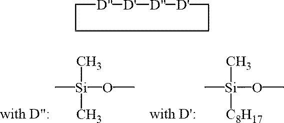

Mention may also be made of mixtures of cyclic polydialkylsiloxanes with organosilicon compounds, such as the mixture of octamethylcyclotetrasiloxane and tetra(trimethylsilyl)pentaerythritol (50/50) and the mixture of octamethylcyclotetrasiloxane and oxy-1,1′-bis(2,2,2′,2′,3,3′-hexatrimethylsilyloxy)neopentane;

(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones falling within this category are also described in the article published in Cosmetics and Toiletries, Vol. 91, Jan. 76, pages 27-32 - Todd & Byers “Volatile Silicone Fluids for Cosmetics”.

Nonvolatile polydialkylsiloxanes, polydialkylsiloxane gums and resins, polyorganosiloxanes modified with the above organofunctional groups, and mixtures thereof, are preferably used.

These silicones are more particularly chosen from polydialkylsiloxanes, among which mention may be made mainly of polydimethylsiloxanes bearing trimethylsilyl end groups. The viscosity of the silicones is measured at 25° C. according to the standard ASTM 445, Appendix C.

Among these polydialkylsiloxanes, mention may be made, in a nonlimiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200, with a viscosity of 60 000 mm²/s;
the Viscasil® oils from General Electric and certain oils of the SF series (SF 96, SF 18) from General Electric.

Mention may also be made of polydimethylsiloxanes bearing dimethylsilanol end groups, known under the name dimethiconol (CTFA), such as the oils of the 48 series from the company Rhodia.

In this category of polydialkylsiloxanes, mention may also be made of the products sold under the names Abil Wax® 9800 and 9801 by the company Goldschmidt, which are poly(C1-C20)dialkylsiloxanes.

The silicone gums that can be used in accordance with the invention are in particular polydialkylsiloxanes and preferably polydimethylsiloxanes with high number-average molecular masses of between 200 000 and 1 000 000, used alone or as a mixture in a solvent. This solvent may be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Products that can be used more particularly in accordance with the invention are mixtures such as:
mixtures formed from a polydimethylsiloxane with a hydroxyl-terminated chain, or dimethiconol (CTFA), and from a cyclic polydimethylsiloxane, also known
as cyclomethicone (CTFA), such as the product Q2 1401 sold by the company Dow Corning;
mixtures of a polydimethylsiloxane gum and a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;
mixtures of two PDMSs with different viscosities, and more particularly of a PDMS gum and a PDMS oil, such as the product SF 1236 from the company General Electric. The product SF 1236 is the mixture of a gum SE 30 defined above with a viscosity of 20 m²/s and of an oil SF 96 with a viscosity of 5 × 10⁻⁶ m²/s. This product preferably includes 15% of gum SE 30 and 85% of an oil SF 96.

The organopolysiloxane resins that can be used in accordance with the invention are crosslinked siloxane systems containing the following units:

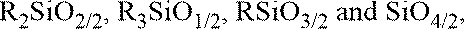

in which R represents an alkyl containing 1 to 16 carbon atoms. Among these products, the ones that are particularly preferred are those in which R denotes a C1-C4 lower alkyl group, more particularly methyl.

Among these resins, mention may be made of the product sold under the name Dow Corning 593 or those sold under the names Silicone Fluid SS 4230 and SS 4267 by the company General Electric, and which are silicones of dimethyl/trimethylsiloxane structure.

Mention may also be made of the trimethyl siloxysilicate-type resins in particular sold under the names X22-4914, X21-5034 and X21-5037 by the company Shin-Etsu.

The organomodified silicones that may be used in accordance with the invention are silicones as defined previously and comprising in their structure one or more organofunctional groups attached via a hydrocarbon-based group.

Besides the silicones described above, the organomodified silicones may be polydiarylsiloxanes, notably polydiphenylsiloxanes, and polyalkylarylsiloxanes functionalized with the organofunctional groups mentioned previously.

The polyalkylarylsiloxanes are chosen particularly from linear and/or branched polydimethyl/methylphenylsiloxanes and polydimethyl/diphenylsiloxanes with a viscosity ranging from 1×10⁻⁵ to 5×10⁻² m²/s at 25° C.

Mention may be made, among these polyalkylarylsiloxanes, by way of examples, of the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

Among the organomodified silicones, mention may be made of polyorganosiloxanes including:
polyethyleneoxy and/or polypropyleneoxy groups optionally comprising C6-C24 alkyl groups, such as the products known as dimethicone copolyol sold by the company Dow Corning under the name DC 1248 or the oils Silwet® L 722, L 7500, L 77 and L 711 from the company Union Carbide, and the (C12)alkyl methicone copolyol sold by the company Dow Corning under the name Q2 5200;
substituted or unsubstituted amino groups, such as the products sold under the names GP 4 Silicone Fluid and GP 7100 by the company Genesee or the products sold under the names Q2 8220 and Dow Corning 929 or 939 by the company Dow Corning. The substituted amino groups are in particular C1-C4 aminoalkyl groups;
alkoxylated groups, such as the product sold under the name Silicone Copolymer F-755 by SWS Silicones, and Abil Wax® 2428, 2434 and 2440 by the company Goldschmidt.

Preferably, the fatty substances do not comprise any C₂-C₃ oxyalkylene units or any glycerol units. Preferably, the fatty substances are not fatty acids and in particular salified fatty acids or soaps which are water-soluble compounds.

The fatty substances are advantageously chosen from C₆-C₁₆ hydrocarbons or hydrocarbons comprising more than 16 carbon atoms, and in particular alkanes, oils of plant origin, fatty alcohols, fatty acid and/or fatty alcohol esters, and silicones, or mixtures thereof.

Preferably, the composition according to the invention comprises a fatty substance chosen from fatty substances that are liquid, at a temperature of 25° C. and at atmospheric pressure.

Preferably, the fatty substances used in the composition according to the invention are non-silicone liquid fatty substances.

Preferably, the liquid fatty substance is chosen from liquid petroleum jelly, C₆-C₁₆ alkanes, volatile linear alkanes, polydecenes, liquid fatty acid and/or fatty alcohol esters, and liquid fatty alcohols, or mixtures thereof.

Even better still, the fatty substance is chosen from liquid petroleum jelly, liquid fatty acid and/or fatty alcohol esters, liquid fatty alcohols, and mixtures thereof, more preferably from liquid petroleum jelly.

The composition according to the invention may also preferentially comprise one or more fatty substances that are solid at a temperature of 25° C. and at atmospheric pressure.

Preferably, the solid fatty substance is chosen from solid fatty acid and/or fatty alcohol esters, solid fatty alcohols, or mixtures thereof.

Preferably, the composition comprises one or more fatty substances chosen from fatty acid and/or fatty alcohol esters, and fatty alcohols, even better still from solid fatty acid and/or fatty alcohol esters, and solid fatty alcohols.

According to one preferred embodiment, the composition of the invention comprises one or more liquid fatty substances and one or more solid fatty substances.

More preferably, the composition comprises one or more liquid fatty substances chosen from liquid petroleum jelly and one or more solid fatty substances chosen from solid fatty acid and/or fatty alcohol esters, and solid fatty alcohols.

The composition has a total content of fatty substance, preferably non-silicone fatty substance, of greater than or equal to 25% by weight, preferably greater than or equal to 30% by weight, and even more preferentially greater than or equal to 40% by weight, relative to the total weight of the composition.

According to a preferred embodiment, the composition has a content of fatty substances, preferably non-silicone fatty substances, that are liquid at 25° C., of greater than or equal to 25% by weight, preferably greater than or equal to 30% by weight, and even more preferentially greater than or equal to 40% by weight, relative to the total weight of the composition.

Preferably, the composition has a content of fatty substances, preferably non-silicone fatty substances, that are liquid at 25° C., ranging from 25% to 80% by weight, more preferentially from 30% to 70% by weight, preferably from 40% to 60% by weight, relative to the total weight of the composition.

In a particularly preferred embodiment, the composition has a content of liquid petroleum jelly of greater than or equal to 25% by weight, more preferentially greater than or equal to 30% by weight, even more preferentially greater than or equal to 40% by weight, relative to the total weight of the composition.

In this embodiment, the composition preferably has a content of liquid petroleum jelly ranging from 25% to 80% by weight, more preferentially from 30% to 70% by weight, preferably from 40% to 60% by weight, relative to the total weight of the composition.

### CHEMICAL OXIDIZING AGENT

The composition according to the invention comprises one or more chemical oxidizing agents. The term “chemical oxidizing agent” is intended to mean an oxidizing agent other than atmospheric oxygen.

More particularly, the chemical oxidizing agent(s) is (are) chosen from hydrogen peroxide, urea peroxide, alkali metal bromates, peroxygenated salts, for instance persulfates or perborates, peracids and precursors thereof, and alkali metal or alkaline-earth metal percarbonates.

Advantageously, this oxidizing agent is hydrogen peroxide.

The concentration of chemical oxidizing agents may range preferably from 0.1% to 20% by weight, and more preferentially from 0.5% to 15% by weight, better still from 1% to 10% by weight relative to the weight of the composition.

### OXIDATION DYE PRECURSOR

As indicated previously, the composition according to the invention may comprise one or more oxidation dye precursors. Preferably, the composition comprises one or more oxidation dye precursors.

As oxidation dye precursors, use may be made of oxidation bases and couplers.

By way of example, the oxidation bases are chosen from para-phenylenediamines, bis(phenyl)alkylenediamines, para-aminophenols, ortho-aminophenols and heterocyclic bases, and addition salts thereof.

Among the para-phenylenediamines, examples that may be mentioned include para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines, examples that may be mentioned include N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof.

Among the para-aminophenols, examples that may be mentioned include para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols, examples that may be mentioned include 2-aminophenol, 2-amino-5-methylphenol, 2-amino-6-methylphenol, 5-acetamido-2-aminophenol and the addition salts thereof.

Among the heterocyclic bases, examples that may be mentioned include pyridine derivatives, pyrimidine derivatives and pyrazole derivatives.

Among the pyridine derivatives, mention may be made of the compounds described for example in patents GB 1 026 978 and GB 1 153 196, for instance 2,5-diaminopyridine, 2-(4-methoxyphenyl)amino-3-aminopyridine and 3,4-diaminopyridine, and the addition salts thereof.

Other pyridine oxidation bases that are useful in the present invention are the 3-aminopyrazolo[1,5-a]pyridine oxidation bases or the addition salts thereof described, for example, in patent application FR 2801308. Examples that may be mentioned include pyrazolo[1,5-a]pyridin-3-ylamine, 2-acetylaminopyrazolo[1,5-a]pyridin-3-ylamine, 2-(morpholin-4-yl)pyrazolo[1,5-a]pyridin-3-ylamine, 3-aminopyrazolo[1,5-a]pyridine-2-carboxylic acid, 2-methoxypyrazolo[1,5-a]pyridin-3-ylamine, (3-aminopyrazolo[1,5-a]pyridin-7-yl)methanol, 2-(3-aminopyrazolo[1,5-a]pyridin-5-yl)ethanol, 2-(3-aminopyrazolo[1,5-a]pyridin-7-yl)ethanol, (3-aminopyrazolo[1,5-a]pyridin-2-yl)methanol, 3,6-diaminopyrazolo[1,5-a]pyridine, 3,4-diaminopyrazolo[1,5-a]pyridine, pyrazolo[1,5-a]pyridine-3,7-diamine, 2-(3-aminopyrazolo[1,5-a]pyridin-2-yloxy)ethanol, 7-(morpholin-4-yl)pyrazolo[1,5-a]pyridin-3-ylamine, pyrazolo[1,5-a]pyridine-3,5-diamine, 5-(morpholin-4-yl)pyrazolo[1,5-a]pyridin-3-ylamine, 2-[(3-aminopyrazolo[1,5-a]pyridin-5-yl)(2-hydroxyethyl)amino] ethanol, 2-[(3-aminopyrazolo[1,5-a]pyridin-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol and 3-aminopyrazolo[1,5-a]pyridin-7-ol, and the addition salts thereof. Salts of 2-(3-aminopyrazolo[1,5-a]pyridin-2-yloxy)ethanol are particularly appreciated.

Among the pyrimidine derivatives, mention may be made of the compounds described, for example, in patents DE 2359399, JP 88-169571, JP 05-63124 and EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof, and the tautomeric forms thereof, when a tautomeric equilibrium exists.

Among the pyrazole derivatives, examples that may be mentioned include 3,4-diaminopyrazole, 4-amino-1,3-dimethyl-5-hydrazinopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof.

Among the couplers that may be used in the composition according to the invention, mention may be made especially of meta-phenylenediamines, meta-aminophenols, meta-diphenols, naphthalene-based couplers, heterocyclic couplers, for instance indole derivatives, indoline derivatives, sesamol and derivatives thereof, pyridine derivatives, pyrazolotriazole derivatives, pyrazolones, indazoles, benzimidazoles, benzothiazoles, benzoxazoles, 1,3-benzodioxoles, quinolines, and the addition salts of these compounds with an acid.

These couplers are more particularly chosen from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis(2,4-diaminophenoxy)propane, sesamol, 1-amino-2-methoxy-4,5-methylenedioxybenzene, α-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c]-1,2,4-triazole and 2,6-dimethylpyrazolo[1,5-b]-1,2,4-triazole, the addition salts thereof with an acid, and mixtures thereof.

The addition salts of the oxidation bases and of the couplers are in particular chosen from the addition salts with an acid such as the hydrochlorides, hydrobromides, sulfates, citrates, succinates, tartrates, lactates, tosylates, benzenesulfonates, phosphates and acetates.

According to one embodiment, the composition comprises at least one oxidation base and optionally a coupler.

When it comprises them, the composition comprises one or more oxidation dye precursors preferably in a total amount ranging from 0.01% to 10% by weight, preferably from 0.1% to 5% by weight, more preferably from 0.3% to 3% by weight relative to the total weight of the composition.

When it comprises them, the composition comprises one or more couplers preferably in a total amount ranging from 0.01% to 10%, preferably from 0.1% to 5% by weight and more preferably from 0.3% to 3% by weight relative to the total weight of the composition.

When it comprises them, the composition comprises one or more oxidation bases preferably in a total amount ranging from 0.01% to 10% by weight, preferably from 0.1% to 5% by weight and more preferably from 0.3% to 3% by weight relative to the total weight of the composition.

### BASIFYING AGENTS

The composition according to the invention may also comprise one or more basifying agents.

The basifying agent may be inorganic or organic or hybrid.

The inorganic basifying agent(s) is (are) preferably chosen from aqueous ammonia, alkali metal carbonates or bicarbonates, sodium hydroxide and potassium hydroxide, or mixtures thereof.

The organic basifying agent(s) is (are) preferably chosen from organic amines with a pKb at 25° C. of less than 12, and preferably less than 10, even more advantageously less than 6. It should be noted that this is the pKb corresponding to the function of highest basicity.

The organic basifying agent(s) is (are) chosen, for example, from alkanolamines, oxyethylenated and/or oxypropylenated ethylenediamines, amino acids and the compounds of formula (A1) below:

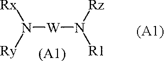

wherein W is a C₁-C₆ alkylene residue optionally substituted with a hydroxyl group or a C₁-C₆ alkyl radical; Rx, Ry, Rz and Rt, which may be identical or different, represent a hydrogen atom or a C₁-C₆ alkyl, C₁-C₆ hydroxyalkyl or C₁-C₆ aminoalkyl radical.

Examples of such amines that may be mentioned include 1,3-diaminopropane, 1,3-diamino-2-propanol, spermine and spermidine.

The term “alkanolamine” is intended to mean an organic amine comprising a primary, secondary or tertiary amine function, and one or more linear or branched C₁-C₈ alkyl groups bearing one or more hydroxyl radicals.

Alkanolamines such as monoalkanolamines, dialkanolamines or trialkanolamines comprising one to three identical or different C₁-C₄ hydroxyalkyl radicals are in particular suitable for performing the invention.

Among compounds of this type, mention may be made of monoethanolamine, diethanolamine, triethanolamine, monoisopropanolamine, diisopropanolamine, N-dimethylaminoethanolamine, 2-amino-2-methyl-1-propanol, triisopropanolamine, 2-amino-2-methyl-1,3-propanediol, 3-amino-1,2-propanediol, 3-dimethylamino-1,2-propanediol and tris(hydroxymethylamino)methane.

More particularly, the amino acids that may be used are of natural or synthetic origin, in their L, D or racemic form, and include at least one acid function chosen more particularly from carboxylic acid, sulfonic acid, phosphonic acid and phosphoric acid functions. The amino acids may be in neutral or ionic form.

As amino acids that can be used in the present invention, mention may in particular be made of aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine and valine.

Advantageously, the amino acids are basic amino acids comprising an additional amine function optionally included in a ring or in a ureido function.

Such basic amino acids are preferably chosen from those corresponding to formula (A2) below:

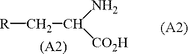

in which R denotes a group chosen from:

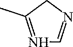

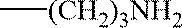

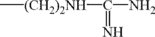

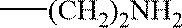

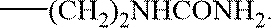

The compounds corresponding to formula (A2) are histidine, lysine, arginine, ornithine and citrulline.

The organic amine may also be chosen from organic amines of heterocyclic type. Besides histidine that has already been mentioned in the amino acids, mention may in particular be made of pyridine, piperidine, imidazole, triazole, tetrazole and benzimidazole.

The organic amine may also be chosen from amino acid dipeptides. As amino acid dipeptides that can be used in the present invention, mention may notably be made of carnosine, anserine and balenine.

The organic amine may also be chosen from compounds comprising a guanidine function. As amines of this type that can be used in the present invention, besides arginine which has already been mentioned as an amino acid, mention may be notably be made of creatine, creatinine, 1,1-dimethylguanidine, 1,1-diethylguanidine, glycocyamine, metformin, agmatine, N-amidinoalanine, 3-guanidinopropionic acid, 4-guanidinobutyric acid and 2-([amino(imino)methyl]amino)ethane-1-sulfonic acid.

Preferably, the organic amine present in the dyeing composition of the invention is an alkanolamine.

Even more preferentially, the organic amine is monoethanolamine.

Hybrid compounds that may be mentioned include the salts of the amines mentioned previously with acids such as carbonic acid or hydrochloric acid.

Guanidine carbonate or monoethanolamine hydrochloride may be used in particular.

Preferably, the composition according to the invention comprises an organic amine, preferably an alkanolamine, and more preferably monoethanolamine.

According to a first particular embodiment, the composition does not contain any aqueous ammonia, or a salt thereof, or the process according to the invention does not use aqueous ammonia, or a salt thereof, as basifying agent.

If, however, according to another particular embodiment, the composition or the process did use any, its content would advantageously not exceed 0.03% by weight (expressed as NH₃) and would preferably not exceed 0.01% by weight, relative to the weight of the composition of the invention. Preferably, if the composition comprises aqueous ammonia, or a salt thereof, then the amount of basifying agent(s) other than the aqueous ammonia is greater than that of the aqueous ammonia (expressed as NH₃).

Advantageously, the total content of basifying agent(s) ranges from 0.01% to 10% by weight, preferably from 0.1% to 5% by weight relative to the total weight of the composition.

According to one preferred embodiment, the total content of organic amine(s) ranges from 0.01% to 10% by weight, preferably from 0.1% to 5% by weight relative to the total weight of the composition.

### THICKENING POLYMERS

The composition according to the invention may also comprise one or more thickening polymers.

Preferably, the thickening polymer(s) may be chosen from cellulose-based thickening polymers (hydroxyethylcellulose, hydroxypropylcellulose, carboxymethylcellulose, quaternized cellulose derivatives such as polyquaternium-67), guar gum and its derivatives (hydroxypropyl guar), gums of microbial origin (xanthan gum, scleroglucan gum), crosslinked homopolymers of acrylic acid or of acrylamidopropanesulfonic acid and associative polymers (polymers comprising hydrophilic regions and hydrophobic regions having a fatty chain (alkyl or alkenyl chain comprising at least 10 carbon atoms) which are capable, in an aqueous medium, of reversibly associating with one another or with other molecules).

More preferably, the thickening polymer(s) may be chosen from cellulose-based thickening polymers (hydroxyethylcellulose, hydroxypropylcellulose, carboxymethylcellulose, quaternized cellulose derivatives such as polyquaternium-67), guar gum and its derivatives such as hydroxypropyl guar and crosslinked homopolymers of acrylic acid.

The content of thickening polymer(s), if they are present, usually ranges from 0.001% to 10% by weight, preferably from 0.01% to 5% by weight relative to the weight of the composition.

### SURFACTANT

The composition according to the present invention may comprise one or more surfactants. These surfactants may be chosen from anionic surfactants, amphoteric or zwitterionic surfactants, cationic surfactants and nonionic surfactants.

The term “anionic surfactant” is intended to mean a surfactant comprising, as ionic or ionizable groups, only anionic groups. These anionic groups are preferably chosen from the following groups: CO₂H, CO₂⁻, SO₃H, SO₃⁻, OSO₃H, OSO₃⁻, H₂PO₃, HPO₃⁻, PO₃²⁻, H₂PO₂, HPO₂⁻, PO₂²⁻, POH and PO⁻.

The amphoteric or zwitterionic surfactant(s) that may be used in the present invention may notably be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

The cationic surfactant(s) that may be used in the composition according to the invention are generally chosen from optionally polyoxyalkylenated primary, secondary or tertiary fatty amine salts, quaternary ammonium salts, and mixtures thereof.

The nonionic surfactant(s) are notably described, for example, in the “Handbook of Surfactants” by M.R. Porter, published by Blackie & Son (Glasgow and London), 1991, pp 116-178. They are especially chosen from fatty alcohols, fatty α-diols, fatty (C₁-C₂₀)alkylphenols or fatty acids, these compounds being ethoxylated, propoxylated or glycerolated and containing at least one fatty chain comprising, for example, from 8 to 18 carbon atoms, the number of ethylene oxide or propylene oxide groups possibly ranging especially from 1 to 200, and the number of glycerol groups possibly ranging especially from 1 to 30.

Mention may also be made of condensates of ethylene oxide and of propylene oxide with fatty alcohols, ethoxylated fatty amides preferably containing from 1 to 30 ethylene oxide units, polyglycerolated fatty amides comprising on average from 1 to 5, and in particular from 1.5 to 4, glycerol groups, ethoxylated fatty acid esters of sorbitan containing from 1 to 30 ethylene oxide units, fatty acid esters of sucrose, fatty acid esters of polyethylene glycol, (C₆-C₂₄ alkyl)polyglycosides, oxyethylenated plant oils, N-(C₆-C₂₄ alkyl)glucamine derivatives, amine oxides such as (C₁₀-C₁₄ alkyl)amine oxides or N-(C₁₀-C₁₄ acyl)aminopropylmorpholine oxides.

Preferably, the surfactant(s) is (are) chosen from nonionic surfactants, preferably from condensates of ethylene oxide and of propylene oxide with fatty alcohols, ethoxylated fatty acid esters of sorbitan containing from 1 to 30 ethylene oxide units and (C₆-C₂₄ alkyl)polyglycosides.

When the composition comprises one or more surfactants, their content may preferably range from 0.05% to 20% by weight, more preferentially from 0.1% to 10% by weight, better still from 0.5% to 5% by weight, relative to the total weight of the composition.

When the composition comprises one or more nonionic surfactants, their content may preferably range from 0.05% to 20% by weight, more preferentially from 0.1% to 10% by weight, better still from 0.5% to 5% by weight, relative to the total weight of the composition.

### CATIONIC POLYMER

The composition according to the invention may also comprise one or more cationic polymers.

As cationic polymer, mention may in particular be made of:

cyclopolymers of alkyldiallylamine or of dialkyldiallylammonium, such as the homopolymers or copolymers including, as main constituent of the chain, units corresponding to formula (III) or (IV):

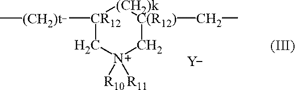

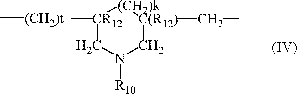

wherein
k and t are equal to 0 or 1, the sum k + t being equal to 1;
R₁₂ denotes a hydrogen atom or a methyl radical;
R₁₀ and R₁₁, independently of one another, denote a C₁-C₆ alkyl group, a C₁-C₅ hydroxyalkyl group, a C₁-C₄ amidoalkyl group or else R₁₀ and R₁₁ may denote, together with the nitrogen atom to which they are attached, a heterocyclic group such as piperidinyl or morpholinyl; R₁₀ and R₁₁, independently of one another, preferably denote a C₁-C₄ alkyl group;
Y⁻ is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer (Polyquaternium-6), for example sold under the name Merquat 100 by the company Nalco. Preferably, the polymers of family (1) are chosen from dialkyldiallylammonium homopolymers.

quaternary diammonium polymers comprising repeating units of formula (V):

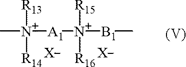

wherein:
R₁₃, R₁₄, R₁₅ and R₁₆, which may be identical or different, represent aliphatic, alicyclic or arylaliphatic radicals comprising from 1 to 20 carbon atoms or C₁-C₁₂ hydroxyalkyl aliphatic radicals;
or else R₁₃, R₁₄, R₁₅ and R₁₆, together or separately, form, with the nitrogen atoms to which they are attached, heterocycles optionally comprising a second non-nitrogen heteroatom;
or else R₁₃, R₁₄, R₁₅ and R₁₆ represent a linear or branched C₁-C₆ alkyl radical substituted with a nitrile, ester, acyl, amide or —CO—O—R₁₇—D or —CO—NH—R₁₇—D group, where R₁₇ is an alkylene and D is a quaternary ammonium group;
A₁ and B₁ represent divalent polymethylene groups comprising from 2 to 20 carbon atoms which can be linear or branched and saturated or unsaturated, and which can contain, bonded to or inserted in the main chain, one or more aromatic rings or one or more oxygen or sulfur atoms or sulfoxide, sulfone, disulfide, amino, alkylamino, hydroxyl, quaternary ammonium, ureido, amide or ester groups; and
X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a linear or branched, saturated or unsaturated alkylene or hydroxyalkylene radical, B₁ may also denote a (CH₂)ₙ—CO—D—OC—(CH₂)ₚ— group with n and p, which may be identical or different, being integers ranging from 2 to 20, and D denoting:
   a) a glycol residue of formula -O-Z-O-, in which Z denotes a linear or branched hydrocarbon-based radical or a group corresponding to one of the following formulae: —(CH₂—CH₂—O)ₓ—CH₂—CH₂— and —[CH₂—CH(CH₃)—O]_{y}—CH₂—CH(CH₃)— where x and y denote an integer from 1 to 4, representing a defined and unique degree of polymerization or any number from 1 to 4 representing an average degree of polymerization;
   b) a bis-secondary diamine residue, such as a piperazine derivative;
   c) a bis-primary diamine residue of formula -NH-Y-NH-, where Y denotes a linear or branched hydrocarbon-based radical, or else the divalent radical —CH₂—CH₂—S—S—CH₂—CH₂—;
   d) a ureylene group of formula -NH-CO-NH-.

Preferably, X⁻ is an anion, such as chloride or bromide. These polymers have a number-average molar mass (Mn) generally of between 1000 and 100 000.

Mention may be made more particularly of cationic polymers that are constituted of repeating units corresponding to the formula (VI):

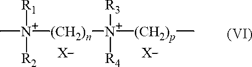

wherein R₁, R₂, R₃ and R₄, which may be identical or different, denote an alkyl or hydroxyalkyl radical containing from 1 to 4 carbon atoms, n and p are integers ranging from 2 to 20, and X⁻ is an anion derived from a mineral or organic acid.

A compound of formula (VI) that is particularly preferred is the one for which R₁, R₂, R₃ and R₄ represent a methyl radical, n = 3, p = 6 and X = Cl, which is known as Hexadimethrine chloride according to the INCI (CTFA) nomenclature.

Preferably, the cationic polymer(s) is/are chosen from dialkyldiallylammonium homopolymers, in particular homopolymers of dimethyldiallylammonium salts, polymers constituted of repeating units corresponding to formula (VI) above, in particular poly(dimethyliminio)-1,3-propanediyl(dimethyliminio)-1,6-hexanediyl dichloride, the INCI name of which is hexadimethrine chloride, and mixtures thereof.

When they are present, the concentration of cationic polymers in the composition according to the present invention may range from 0.01% to 10% by weight relative to the weight of the composition, preferably from 0.1% to 5% relative to the weight of the composition, even more advantageously from 0.015% to 3% by weight relative to the weight of the composition.

The composition according to the invention is preferably aqueous. When it is aqueous, the composition according to the invention comprises water in a content preferably ranging from 20% to 80% by weight, better still from 25% to 70% by weight, even better still from 30% to 60% by weight relative to the total weight thereof.

The pH of the composition is preferably greater than or equal to 7.5. The pH may range from 7.5 to 11, preferably from 8 to 10.5, more preferably from 8.5 to 10 and preferentially from 9 to 10.

### ADDITIVES

The composition according to the present invention may also optionally comprise one or more additives, different from the compounds of the invention and among which mention may be made of nonionic, anionic or amphoteric polymers or mixtures thereof other than thickening polymers, antidandruff agents, anti-seborrhoea agents, vitamins and provitamins including panthenol, sunscreens, mineral or organic pigments, sequestrants, plasticizers, solubilizers, acidifying agents, opacifiers or pearlescent agents, antioxidants, hydroxy acids, fragrances, preserving agents and pigments.

Of course, those skilled in the art will take care to choose this or these optional additional compounds so that the advantageous properties intrinsically associated with the composition according to the invention are not, or not substantially, detrimentally affected by the envisioned addition(s).

The above additives can generally be present in an amount of, for each of them, between 0% and 20% by weight, relative to the total weight of the composition.

### PROCESS OF THE INVENTION

The composition according to the invention as previously defined is applied to dry or wet keratin fibers. It is left in place on the fibers for a time generally of from 1 minute to 1 hour, preferably from 5 minutes to 30 minutes.

The temperature during the coloring process is conventionally between room temperature (between 15° C. and 25° C.) and 80° C., preferably between room temperature and 60° C.

After the treatment, the human keratin fibers are optionally rinsed with water, optionally washed with a shampoo and then rinsed with water, before being dried or left to dry.

The composition according to the invention is generally prepared by mixing at least two compositions.

In particular, the composition according to the invention results from the mixing of two compositions, comprising the ingredients as defined previously distributed in the following manner:
a composition (A) comprising one or more anthraquinone cationic direct dyes and one or more basifying agents and/or one or more oxidation dye precursors preferably one or more oxidation dye precursors;
a composition (B) comprising one or more chemical oxidizing agents;
it being understood that at least one of the compositions (A) or (B) comprises one or more fatty substances, preferably one or more liquid fatty substances, in a content such that the content of fatty substance in the mixture of compositions (A) and (B) is at least 25% by weight, preferably at least 30% by weight, more preferably at least 40% by weight relative to the total weight of the mixture of compositions (A) and (B).

Preferably, composition (A) comprises at least 30% by weight of one or more fatty substances relative to the total weight of composition (A).

Preferentially, at least one of the compositions (A) or (B) is aqueous. Preferably, composition (A) is aqueous.

Even more preferentially, both the compositions (A) and (B) are aqueous.

According to another variant of the invention, the composition according to the invention results from the mixing of three compositions, comprising the ingredients as defined previously distributed in the following manner:
a composition (A1) comprising one or more anthraquinone cationic direct dyes;
a composition (A2) comprising one or more basifying agents and/or one or more oxidation dye precursors, preferably one or more oxidation dye precursors;
a composition (B) comprising one or more chemical oxidizing agents;
it being understood that at least one of the compositions (A1), (A2) or (B) comprises one or more fatty substances, preferably one or more liquid fatty substances, in a content such that the content of fatty substance in the mixture of compositions (A1), (A2) and (B) is at least 25% by weight, preferably at least 30% by weight, more preferably at least 40% by weight relative to the total weight of the mixture of compositions (A1), (A2) and (B).

Preferably, composition (A2) comprises at least 30% by weight of one or more fatty substances relative to the total weight of composition (A2).

Preferentially, at least one of the compositions (A1), (A2) or (B) is aqueous. Preferably, composition (A2) is aqueous.

Even more preferentially, both the compositions (A2) and (B) are aqueous.

Even more preferentially, all three compositions (A1), (A2) and (B) are aqueous.

### DEVICES

Another subject of the invention is a multi-compartment device.

According to a first embodiment, the device of the invention comprises:
a first compartment containing a composition (A) comprising:
   one or more anthraquinone cationic direct dyes, and
   one or more oxidation dye precursors and/or one or more basifying agents, preferably one or more oxidation dye precursors,
a second compartment containing a composition (B) comprising one or more chemical oxidizing agents,
it being understood that at least one of the compositions (A) or (B) comprises at least one fatty substance as defined above, in a content such that the content of fatty substance in the mixture of compositions (A) and (B) is at least 25% by weight, preferably at least 30% by weight, more preferably at least 40% by weight relative to the total weight of the mixture of compositions (A) and (B), the compositions of the compartments being intended to be mixed before application.

According to a second embodiment, the device of the invention comprises:
a first compartment containing a composition (A1) comprising one or more anthraquinone cationic direct dyes;
a second compartment containing a composition (A2) comprising one or more basifying agents and/or one or more oxidation dye precursors, preferably one or more oxidation dye precursors;
a third compartment containing a composition (B) comprising one or more chemical oxidizing agents,
it being understood that at least one of the compositions (A1), (A2) or (B) comprises one or more fatty substances, preferably one or more liquid fatty substances, in a content such that the content of fatty substance in the mixture of compositions (A1), (A2) and (B) is at least 25% by weight, preferably at least 30% by weight, more preferably at least 40% by weight relative to the total weight of the mixture of compositions (A1), (A2) and (B), the compositions of the compartments being intended to be mixed before application.

The examples that follow serve to illustrate the invention without, however, being limiting in nature.

### EXAMPLES

In the examples that follow, all the amounts are given as weight percentage of active material (AM) relative to the total weight of the composition, unless otherwise specified.

### Example 1

Compositions A and B were prepared from the ingredients whose contents are indicated in the table below:

TABLE 1

| | | |
|---|---|---|
| | A (invention) | B (comparative) |
| LIQUID PARAFFIN | 60 | 10 |
| DECETH-5 | 1.2 | 1.2 |
| CETYL PALMITATE | 2 | 2 |
| C20-22 ALCOHOLS | 4.6 | 4.6 |
| OLETH-20 | 4 | 4 |
| OLETH-10 | 1 | 1 |
| GLYCEROL | 5 | 5 |
| CARBOMER | 0.1 | 0.1 |
| ASCORBIC ACID | 0.12 | 0.12 |
| EDTA | 0.2 | 0.2 |
| CETEARETH-60 MYRISTYL GLYCOL | 0.01 | 0.01 |
| ETHANOLAMINE | 4.4 | 4.4 |
| HC BLUE NO. 16 | 2.5 | 2.5 |
| WATER | qs 100 | qs 100 |

TABLE 2

| | |
|---|---|
| | Oxidizing composition C |
| HYDROGEN PEROXIDE | 6 |
| CETEARYL ALCOHOL | 6 |
| HEXADIMETHRINE CHLORIDE | 0.15 |
| POLYQUATERNIUM-6 | 0.2 |
| MINERAL OIL | 20 |
| TETRASODIUM ETIDRONATE | 0.06 |
| PEG-4 RAPESEEDAMIDE | 1.2 |
| GLYCEROL | 0.5 |
| STEARETH-20 | 5 |
| TOCOPHEROL | 0.1 |
| TETRASODIUM PYROPHOSPHATE | 0.04 |
| pH Agent | qs pH 2.2 |
| WATER | qs 100 |

At the moment of use, each of compositions A and B is mixed with one times its weight of the oxidizing composition C in order to obtain, respectively, the compositions M1 (A+C) and M2 (B+C).

### Measurement of the Viscosity of the Compositions

The viscosity is measured by means of a Rheomat RM 180 rheometer (200 rpm, 22° C., measurement at 30 s, spindle 3 for compositions M1 and M2).

The results obtained are as follows (expressed in mPa.s):

TABLE 3

| | | |
|---|---|---|
| | Composition M1 (invention) | Composition M2 (comparative) |
| Viscosity | 2235 mPa.s | 580 mPa.s |

Composition M1 according to the invention has a higher viscosity than the comparative composition M2. Thus, the composition according to the invention is easy to use.

Since composition M1 according to the invention has a thicker texture compared to the comparative composition M2, it has a lower risk of running during application on the head than the comparative composition.

### Dyeing Results

Compositions M1 and M2 are applied to locks of permanent-waved 90% white natural hair (PWW) and to locks of highly sensitized hair (SA40) in a proportion of 3 g of composition per gram of lock of hair.

The locks were then placed on hotplates thermostatically regulated at 27° C. After a leave-on time of 30 minutes, the locks are rinsed, washed and dried.

The coloration of the locks of hair is evaluated in the L*a*b* system, with a MINOLTA CM2002 ® spectrophotometer.

The selectivity of the coloration is the variation in color between natural hair and permanent-waved hair. The natural hair is representative of the nature of the hair at the root whereas the permanent-waved hair is representative of the nature of the hair at the end.

The selectivity is measured by ΔE which is the variation in color between the natural hair and the permanent-waved hair, ΔE being obtained from the formula:

$$Δ \text{E=} \sqrt{{\left(\text{L*} - {\text{L}}_{\text{o}} *\right)}^{2} + {\left(\text{a*} - {\text{a}}_{\text{o}} *\right)}^{2} + {\left(\text{b*} - {\text{b}}_{\text{o}} *\right)}^{2}}$$

wherein L* represents the intensity and a* and b* represent the chromaticity of the dyed natural hair, and L₀^{∗} represents the intensity and a₀^{∗} and b₀^{∗} represent the chromaticity of the dyed permanent-waved hair. The lower the value of ΔE, the lower the selectivity and the more uniform the coloration along the hair.

The results are as follows:

TABLE 4

| | Type of hair | L* | a* | b* | ΔE |
|---|---|---|---|---|---|
| M1 | PWW | 26.69 | 2.81 | -32.23 | 10.42 |
| | SA40 | 20.62 | 7.65 | -25.28 | |
| M2 | PWW | 27.75 | 5.56 | -34.99 | 15.28 |
| | SA40 | 19.31 | 8.58 | -22.61 | |

Composition M1 according to the invention leads to a lower value of ΔE, thus to a better selectivity, compared to the comparative composition M2.

### Study of the Color of the Rinsing Water (Bleeding Test)

Compositions M1 and M2 were applied to locks of natural white hair in a proportion of 5 g of composition per gram of hair. At the end of a leave-on time of 15 min, the hair is rinsed. It is observed that the color of the rinsing water for the locks dyed with composition M1 is lighter than that of the locks dyed with composition M2: there is less bleeding of the color with composition M1 according to the invention compared to composition M2.

The locks are then washed with a standard shampoo. It is again observed that the color of the rinsing water for the locks dyed with composition M1 is lighter than that of the locks dyed with composition M2 after one shampoo wash: there is less bleeding of the color with composition M1 according to the invention compared to composition M2 after one shampoo wash.

### Example 2

Compositions A′ can be prepared from the ingredients whose contents are indicated in the table below:

| | |
|---|---|
| | Composition A′ |
| 2,4-DIAMINOPHENOXYETHANOL HCL | 0.05 |
| N,N-BIS(2-HYDROXYETHYL)-p-PHENYLENEDIAMINE SULFATE | 0.045 |
| 6-HYDROXYINDOLE | 0.04 |
| 2-AMINO-3-HYDROXYPYRIDINE | 0.036 |
| HYDROXYBENZOMORPHOLINE | 0.28 |
| HYDROXYETHYL-3,4-METHYLENEDIOXYANILINE HCL | 0.15 |
| m-AMINOPHENOL | 0.13 |
| TOLUENE-2,5-DIAMINE | 0.53 |
| HC BLUE 16 | 0.1 |
| MINERAL OIL | 60 |
| ETHANOLAMINE | 4.43 |
| DECYL GLUCOSIDE | 1.59 |
| SODIUM LAURYL SULFATE | 1.24 |
| PEG-40 HYDROGENATED CASTOR OIL | 1 |
| DISODIUM COCOAMPHODIACETATE | 0.94 |
| HYDROXYPROPYL GUAR | 0.3 |
| SODIUM METABISULFITE | 0.22 |
| EDTA | 0.2 |
| ASCORBIC ACID | 0.12 |
| HELIANTHUS ANNUUS (SUNFLOWER) SEED OIL | 0.1 |
| CAMELLIA OLEIFERA SEED OIL | 0.1 |
| FRAGRANCE | 0.95 |
| WATER | Qsp 100 |

At the moment of use, composition A′ can be mixed with one times its weight of the oxidizing composition C. The mixed composition can be applied on white hair to obtain light chestnut ash hair.

1-19. (canceled)

20. A composition for dyeing and/or lightening human keratin fibers, comprising:
one or more anthraquinone cationic direct dyes,
at least 25% by weight of one or more fatty substances relative to the total weight of the composition,
one or more chemical oxidizing agents, and
one or more basifying agents and/or one or more oxidation dye precursors.

21. The composition of claim 20, wherein the one or more anthraquinone cationic direct dyes are chosen from the cationic dyes of formula (I): 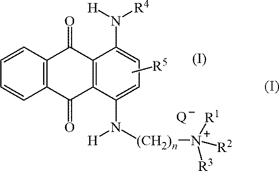
wherein:
R¹, R² and R³ are independently chosen from a hydrogen atom or an optionally substituted (C₁-C₈)alkyl group;
R⁴ represents a hydrogen atom or an optionally substituted (C₁-C₈)alkyl group;
R⁵ represents a hydrogen atom, an optionally substituted (C₁-C₈)alkyl group, an optionally substituted (C₁-C₈)alkylene group, a halogen, a hydroxyl group or a (C₁-C₈)alkoxy group;
n represents a number between 1 and 8; and
Q⁻ represents an organic or mineral anionic counterion.

22. The composition of claim 20, wherein the one or more anthraquinone cationic direct dyes are chosen from the cationic dyes of formula (I′) or (I″): 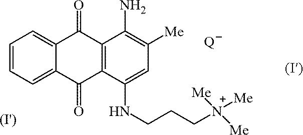 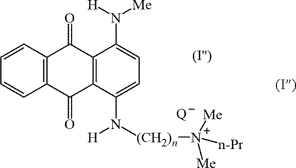
wherein Q⁻ represents an anionic counterion.

23. The composition of claim 20, wherein the one or more anthraquinone cationic direct dyes are chosen from HC Blue 16, HC Blue 17, or mixtures thereof.

24. The composition of claim 20, wherein the total amount of anthraquinone cationic direct dye ranges from 0.01% to 15% by weight, relative to the total weight of the composition.

25. The composition of claim 20, wherein the total amount of fatty substances is greater than or equal to 30% by weight, relative to the total weight of the composition.

26. The composition of claim 20, wherein the one or more fatty substances are chosen from compounds that are liquid or pasty at room temperature and at atmospheric pressure.

27. The composition of claim 20, wherein the one or more fatty substances are chosen from non-silicone fatty substances.

28. The composition of claim 20, wherein the one or more fatty substances are chosen from C₆-C₁₆ hydrocarbons, hydrocarbons comprising more than 16 carbon atoms, non-silicone oils of animal origin, triglycerides of plant or synthetic origin, fatty alcohols, and fatty acid and/or fatty alcohol esters, or mixtures thereof.

29. The composition of claim 20, wherein the one or more fatty substances are chosen from liquid petroleum jelly or liquid paraffin, polydecenes, fatty acid and/or fatty alcohol esters that are liquid at room temperature and at atmospheric pressure, fatty alcohols that are liquid at room temperature and at atmospheric pressure, or mixtures thereof.

30. The composition of claim 20, wherein the one or more chemical oxidizing agents are chosen from hydrogen peroxide, urea peroxide, alkali metal bromates, peroxygenated salts, or mixtures thereof.

31. The composition of claim 20, wherein the total amount of chemical oxidizing agents ranges from 0.1% to 20% by weight, relative to the total weight of the composition.

32. The composition of claim 20, comprising one or more basifying agents chosen from organic amines.

33. The composition of claim 20, wherein the total amount of the one or more basifying agents ranges from 0.01% to 10% by weight, relative to the total weight of the composition.

34. The composition of claim 20, wherein the one or more oxidation dye precursors are chosen from oxidation bases and couplers.

35. The composition of claim 20, wherein the total amount of the one or more oxidation dye precursors ranges from 0.01% to 10% by weight, relative to the total weight of the composition.

36. A multi-compartment device comprising:
a first compartment containing a composition (A) comprising:
one or more anthraquinone cationic direct dyes, and
at least one of one or more oxidation dye precursors or one or more basifying agents;
a second compartment containing a composition (B) comprising one or more chemical oxidizing agents,
wherein at least one of composition (A) or composition (B) comprises at least one fatty substance in an amount of at least 25% by weight, relative to the total weight of composition (A) and composition (B), and
wherein composition (A) and composition (B) are mixed before application.

37. A process for dyeing and/or lightening human keratin fibers comprising applying to the fibers a composition comprising:
one or more anthraquinone cationic direct dyes,
at least 25% by weight of one or more fatty substances relative to the total weight of the composition,
one or more chemical oxidizing agents, and
one or more basifying agents and/or one or more oxidation dye precursors.

38. The process of claim 37, wherein the composition is formed by mixing composition (A) with composition (B), wherein:
composition (A) comprises:
one or more anthraquinone cationic direct dyes, and
at least one of one or more oxidation dye precursors or one or more basifying agents;
composition (B) comprises:
one or more chemical oxidizing agents; and
at least one of composition (A) or composition (B) comprises at least one fatty substance in an amount of at least 25% by weight, relative to the total weight of the mixture of composition (A) and composition (B).

39. The process of claim 37, wherein the composition is formed by mixing composition (A1), composition (A2), and composition (B), wherein:
composition (A1) comprises one or more anthraquinone cationic direct dyes;
composition (A2) comprises at least one of one or more basifying agents or one or more oxidation dye precursors;
composition (B) comprises one or more chemical oxidizing agents; and
at least one of composition (A1), composition (A2), or composition (B) comprises one or more fatty substances in an amount of at least 25% by weight, relative to the total weight of the mixture of composition (A1), composition (A2), and composition (B).
